(19) United States
(12) Patent Application Publication
(10) Pub. No.: US 2019/0223570 A1
(43) Pub. Date: Jul. 25, 2019

(54) Eye contact case and method of use

(71) Applicant: Abraham Sharoni, New York, NY (US)

(72) Inventor: Abraham Sharoni, New York, NY (US)

(21) Appl. No.: 15/995,828

(22) Filed: Jun. 1, 2018

Related U.S. Application Data
(60) Provisional application No. 62/619,264, filed on Jan. 19, 2018.

(51) Int. Cl. A45C 11/00; A61L 12/08; G02C 13/00

(52) U.S. Cl. CPC A45C 11/005; B65D 2585/545; G02C 13/008; A61L 12/086

(57) ABSTRACT

A contact lens case with a plurality of sections configured to conjoin to each other. The sections each contain one or more hemispherical wells that align to form a spherical cavity to hold the contact lens therein when the sections are conjoined. The cavity may also be an ovoid, globular or a flattened sphere. The case may also include a first magnet to couple the first section to the second section, where the second section may have a second magnet or a magnetic material in a location that corresponds to the first magnet.

### PRIORITY CLAIM/INCORPORATION BY REFERENCE

This application claims priority to U.S. provisional application 62/619,264 filed Jan. 16, 2018 entitled “Eye contact case and method of use” which is incorporated herein, its entirety, by reference.

### FIELD

The present invention relates to contact lens cases, and more specifically to a contact lens case that has cavities or wells having a shape (e.g., spherical, ovoid, etc.) that hold the contacts therein, but do not cause the contacts to lay against a sharp edge or corner or cause the contacts to fold over as the orientation of contact lens case changes.

### BACKGROUND INFORMATION

Contacts lens cases have sides that may join at sharp angles or be completely flat. When the orientation of these cases changes, the lens inside can settle resting against these sharp angles or flat surfaces. In some instances, this settling against the sharp angles or flat surfaces causes the contact lens to conform to a shape that is not the shape of the contact lens, while in other cases, the sharp angles or flat surfaces cause the contact lens to fold over onto itself. Thus, the shapes of the conventional contact lens cases compromise the integrity and life of the lens.

### SUMMARY

In an exemplary embodiment described herein, a contact lens case includes a body having two sections configured to secure to each other. The two sections each contain two hemispherical wells that fluidly seal to form a spherical cavity when the two sections are secured.

In another exemplary embodiment, the cavity may be one of an ovoid shape, a globular shape or a flattened sphere shape.

An exemplary contact lens case may also have a first magnet to couple the first section to the second section. The second section may have a second magnet or a magnetic material in a location that corresponds to the first magnet of the first section to couple the first section to the second section. The first magnet may include a plurality of magnets. The first magnet may be arranged to have a surface of the magnet be flush with a surface of the first section that comes in contact with a surface of the second section when the first and second sections are coupled.

The first and second sections may be coupled to fluidly seal the cavity. An exemplary contact lens case may include a gasket for fluidly sealing the cavity and/or a surface treatment to prevent leakage.

The exemplary contact lens case may also include a hinge coupling the first and second sections, the hinge allowing the first and second sections to move between an open position and a closed position.

### BRIEF DESCRIPTION OF THE DRAWINGS

FIG. 1 shows a view of an interior surface of a first section of an exemplary contact lens case according to various embodiments described herein.

FIG. 2 shows a perspective view of a second section of the exemplary contacts case according to the various embodiments described herein.

FIG. 3 shows a perspective view of both sections of the exemplary contacts case according to the various embodiments described herein.

FIG. 4 shows a transparent perspective view of both sections of the exemplary contacts lens case oriented in a closed position according to various embodiments described herein.

### DETAILED DESCRIPTION

The exemplary embodiments may be further understood with reference to the following description and the related appended drawings, wherein like elements are provided with the same reference numerals. It is noted that in this description, the contact lenses may be referred to as a lens, a contact, a contact lens, in the singular or plural. The exemplary embodiments describe a contact lens case including a body forming two sections configured to engage with each other. In some exemplary embodiments the two sections are configured to engage with each other via a plurality of magnets; however, alternative embodiments may include other quick-release devices such as snaps, clips, hook-loop and/or other devices configured to achieve the same result. The body forms two cavities configured to fluidly seal contact solution along with the contact therein. Removing the two sections from each other provides access to the two cavities. In some exemplary embodiments, a surface treatment may be used within the inner surface of the cavities or wells; however, it will also be appreciated that the inner surface may be smooth.

The exemplary embodiments include upper and lower body sections that include hemispherical wells that when joined together, form a spherical cavity. As will be described in greater detail below, each body section will include one or more wells. When these body sections are coupled, the corresponding wells will form a cavity. The spherical cavities retain the contacts such that, regardless of the orientation of the case, the lens rests against a surface that contours to its shape so the integrity of the lens is maintained throughout its lifespan. It should be understood that the exemplary embodiments do not require a perfectly spherical shape, but may include other shapes that replicate the contour of the contact lens. For example, the cavity may be ovoid, globular, have minor flat areas such as a flattened sphere, etc. The point of the shape being that the cavity will include no sharp corners or edges against which the contact lens may lay or cause the contact lens to fold over as the orientation of the contact lens case changes.

FIG. 1 shows a top view of an interior surface of section 6 of an exemplary contact lens case 100 according to various exemplary embodiments described herein. The section 6 includes a first hemispherical well 1 and a second hemispherical well 2. It is noted that the exemplary embodiments include two hemispherical wells that will typically correspond to a right eye contact lens and a left eye contact lens. However, it is possible to have a contact lens case that includes more or less wells. The section 6 further includes a flat surface 5. As will become apparent from the below description, the flat surface 5 of section 6 will be coupled to a flat surface of a corresponding section to form the complete contact lens case 100, where the hemispherical wells 1 and 2 will be adjacent to corresponding hemispherical wells for the corresponding section to form spherical cavities for holding the contact lenses and solution.

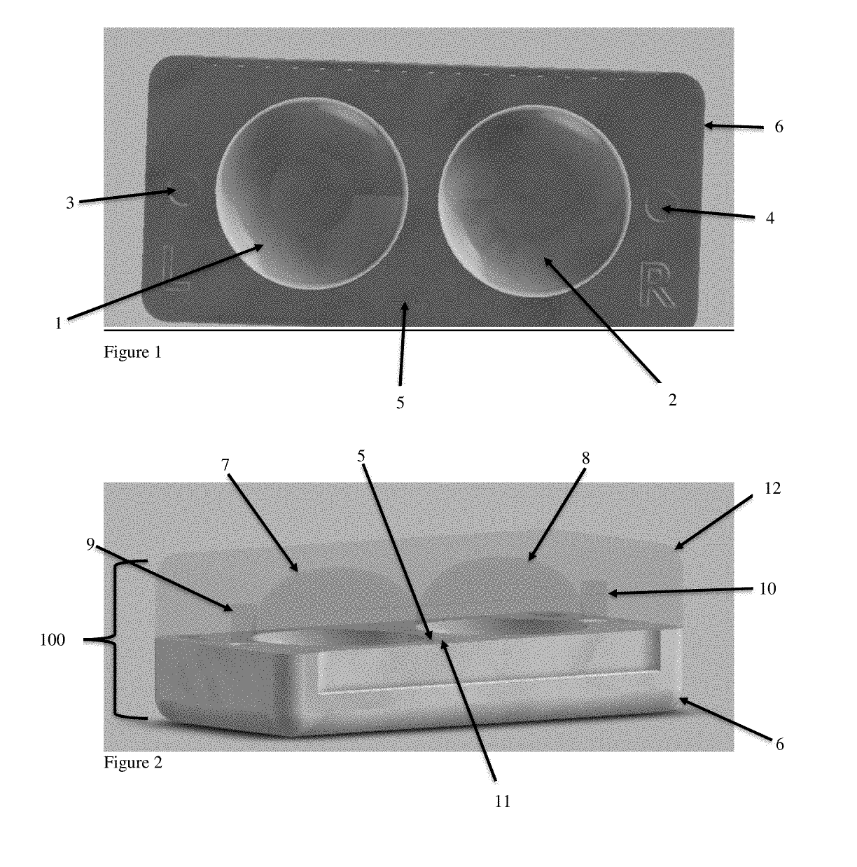

FIG. 2 shows a second section 12 of the contact lens case 100 resting on the previously described section 6. In the present embodiment the two sections have the same dimensions and, when joined, the exterior surfaces are flush. The interior surface 5 of section 6 has the same dimensions as the interior surface 11 of the section 12. The left hemispherical well 7 of the section 12 is the same distance from all corresponding edges of the interior surface 11 of the section 12 as the left hemispherical well 1 of the section 6 is to the interior surface 5 of the section 6. Likewise, the right hemispherical well 8 of the section 12 sits and equal distance from all edges of the interior surface 11 of the section 12 as the right hemispherical well 2 of the section 6 does to the interior surface 5 of the section 6. This is also true of the recessed magnets within both sections. The left recessed magnet 3 of the section 6 is placed and equal distance from all the edges of the interior surface 5 of the section 6 as the left recessed magnet 9 is to all the edges of the interior surface 11 of the section 12. The right recessed magnet 4 of the section 6 is placed an equal distance from all the edges of the interior surface 5 of the section 6 as the right recessed magnet 10 is to all the edges of the interior surface 11 of the section 12. It is noted that the hemispherical wells and the magnets are referred to as left and right, but this is just for ease of description when referring to the exemplary embodiments shown in the figures. There is no requirement for a specific orientation or number of the hemispherical wells or magnets.

In the present embodiment the wells are placed symmetrically within their respective sections, each being equal distance from the exterior edges of the section. This does not have to be the case, the wells may be placed anywhere within the surface of their respective sections so long as the corresponding well of the other section aligns to form the spherical cavity when the sections are coupled. Moreover, the two sections 6 and 12 are not required to have the same exterior dimensions. One of the sections 6 or 12 may be larger or have a different exterior shape than the other section 6 or 12. As described above, the sections 6 and 12 should have corresponding hemispherical wells configured to be adjacent to each other to form the spherical cavity when the sections 6 and 12 are coupled and corresponding magnets to seal or couple the two sections 6 and 12 together. In addition, the left and right sides of the case do not have to be the same shape so long as the hemispherical wells align when the case is in a closed position. Furthermore, with respect to the magnets, it is also possible that only one of the sections 6 or 12 has magnets, while the other section 6 or 12 merely has a magnetic material (iron, steel, nickel, etc.) on the interior surface to which the magnets of the other section may adhere. For example, the section 6 may have the described magnets 3 and 4, while the section 12 may have a stainless steel interior surface 11 allowing the magnets 3 and 4 to adhere to the interior surface 11 when the sections 6 and 12 are joined.

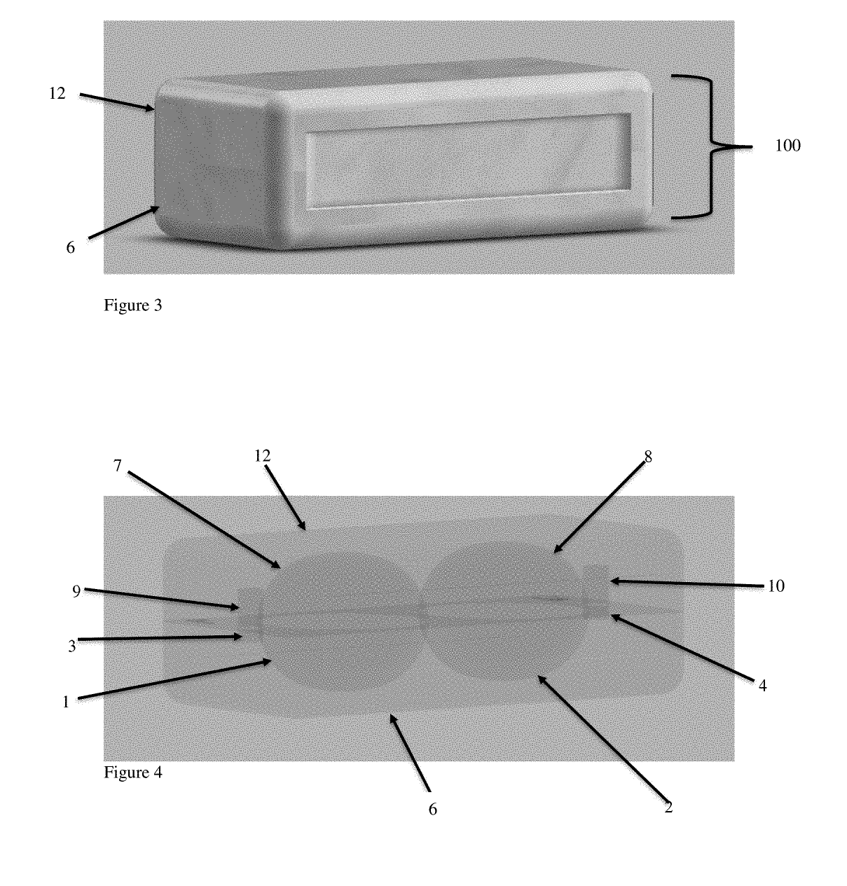

FIG. 3 shows a perspective view of both sections 6 and 12 of the exemplary contacts lens case 100 according to the various embodiments described herein. FIG. 3 shows the exterior of the contacts lens case 100 when the two sections 6 and 12 have been coupled. The seams where the sections 6 and 12 meet are flush to prevent any edges from catching and pulling the case open. Each section 6 and 12 includes an exterior ridge 13, 14, respectively, to allow gripping the contact lens case 100 and pulling it apart easier for the user. When coupled this ridge creates a rectangular shape on the surface of the case 100. This ridge may be any shape on the exterior of the case or may not be there at all.

FIG. 4 shows a transparent perspective view of both sections 6 and 12 of the exemplary contacts lens case 100 oriented in a closed position according to various embodiments described herein. FIG. 4 shows a transparent view of the contacts lens case 100 where the two sections are coupled such as shown in FIG. 3. The interior surface 11 of section 12 is placed on top of the interior surface 5 of section 6 and the corresponding left magnets 3 and 9 and right magnets 4 and 10 align to keep the sections 6 and 12 together to close and seal the contact lens case 100. When the contact lens case 100 is closed the left hemispherical well 1 of section 6 aligns with the left hemispherical well 7 of section 12 creating a spherical cavity that will hold a contact lens and contact lens liquid. The same is true of the right hemispherical well 2 of section 6 and the right hemispherical well 10 of section 12.

A typical use of the contact lens case 100 may be described as follows: The section 6 may be placed on a flat surface and a user may place contact lens fluid and a contact lens into each of the hemispherical wells 1 and 2. The user may then close the contact lens case 100 by coupling the section 12 to the section 6, thereby aligning the hemispherical wells and magnets as described above. This coupling forms a fluidly sealed connection between the sections 6 and 12 such that the contact lens fluid is retained within the spherical cavities in any orientation of the contact lens case 100. The user may then place the contact lens case 100 into their pocket, a purse, a piece of luggage, a briefcase, etc. As the user moves (or the item into which the contact lens case 100 was placed moves), the contact lens case 100 may move or shift into various orientations. As the contact lens case 100 moves, the contact lens and contact lens fluid may move around freely within the spherical cavities. As described above, since the spherical cavities do not have any sharp edges, corners or angles, the contact lens will not rest against any of these types of surfaces and also will not fold over onto itself as the contact lens case 100 is jostled or otherwise moved. When the user is ready to use the contact lenses, the user may decouple the sections 6 and 12 by pulling the sections apart, e.g., using the ridges 13 and/or 14, to gain access to the contact lenses.

As described above, when the sections 6 and 12 are coupled, the spherical cavities are fluidly sealed to keep the contact lens fluid from leaking out of the contact lens case 100. There may be other components that aid in forming this seal. For example, each of the hemispherical wells 1, 2, 7, 8 may have a gasket or other seal that engages with the corresponding gasket of the hemispherical well to seal in the fluid. The gasket or seal may be made of any type of material typically used for gaskets, e.g., paper, rubber, silicone, metal, cork, felt, neoprene, nitrile rubber, fiberglass, polytetrafluoroethylene (PTFE), plastic polymer, etc. Other fluidly sealing manners may also be used, such as male and female mating parts, etc.

It is also noted that the sections 6 and 12 are shown as being completely separable. However, the sections 6 and 12 may also be coupled in a manner that does not make them completely separable. For example, one edge of the sections 6 and 12 may include a hinge that allows the sections 6 and 12 to be separated to gain access to the wells, but not come completely apart so that a user cannot lose one section. This type of coupling may also ensure that when the user closes the case, the wells and the magnets are properly aligned.

The interior of the wells of sections 6 and 12 may be treated with various compounds to assure that the material used for the case does not react with the contact liquid used in the case or the contact itself. The interior surfaces 5 and 11 may also be treated with compounds to create a hydrophobic around the wells. This would help keep contacts lens liquid in the wells and prevent liquid from seeping in between surfaces 5 and 11.

The materials used to make the case may range from, but are not limited to, injection mold plastic to man-made stone. Any material that will hold the spherical shape may be used to craft the contacts lens case.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

1. A contact lens case, comprising:
a first section comprising a first rectangular prism having a first section first surface including a first hemispherical well that concaves into the first section without protruding from the first section first surface, the first section further comprising a first section second surface including a first ridge; and
a second section comprising a second rectangular prism having a second section first surface including a second hemispherical well that concaves into the second section without protruding from the second section first surface, the second section further comprising a second section second surface comprising a second ridge,
wherein the first and second sections are configured to be coupled to form a third rectangular prism that is a combination of the first and second rectangular prisms,
wherein the first section first surface and second section first surface abut in an interior of the third rectangular prism such that the first hemispherical well corresponds to the second hemispherical well to form a cavity to hold a contact lens,
wherein the cavity comprises one of a spherical shape, an ovoid shape, a globular shape, or a flattened sphere shape, and
wherein the first section second surface and the second section second surface form a continuous exterior surface of the third rectangular prism and the first ridge of the first section second surface and the second ridge of the second section second surface are configured to be used to open the third rectangular prism by separating the first and second sections.

2. (canceled)

3. (canceled)

4. The contact lens case of claim 1, wherein the first section further comprises:
a first magnet to couple the first section to the second section.

5. The contact lens case of claim 4, wherein the second section comprises one of a second magnet or a magnetic material in a location that corresponds to the first magnet of the first section to couple the first section to the second section.

6. The contact lens case of claim 4, wherein the first magnet is a plurality of magnets.

7. The contact lens case of claim 4, wherein the first magnet is arranged to have a surface of the magnet be flush with a surface of the first section that comes in contact with a surface of the second section when the first and second sections are coupled.

8. (canceled)

9. (canceled)

10. The contact lens case of claim 1, wherein the first hemispherical well comprises a plurality of wells and the second hemispherical well comprises a corresponding plurality of wells.

11. The contact lens case of claim 1, further comprising a hinge coupling the first and second sections, the hinge allowing the first and second sections to move between an open position and a closed position.

12. (canceled)

13. The contact lens case of claim 1, wherein the first hemispherical well includes a first gasket and the second hemispherical well includes a second gasket.

14. The contact lens case of claim 13, wherein, when the first and second sections are coupled, the first gasket and the second gasket fluidly seal the cavity.
